(19) United States
(12) Patent Application Publication
(10) Pub. No.: US 2016/0158274 A1
(43) Pub. Date: Jun. 9, 2016

(54) COMPOSITION FOR MUCOUS DISPERSION OR HYDRATION, CONTAINING POLY-r-GLUTAMIC ACID

(71) Applicants: BIOLEADERS CORPORATION, Daejeon (KR); THE INDUSTRY AND ACADEMIC COOPERATION IN CHUNGNAM NATIONAL UNIVERSITY, Daejeon (KR)

(72) Inventors: Il-Han Lee, Daejeon (KR); Yong Taik Lim, Daejeon (KR)

(21) Appl. No.: 14/894,389

(22) PCT Filed: May 27, 2014
(86) PCT No.: PCT/KR2014/004688

§ 371 Date: Feb. 24, 2016

(30) Foreign Application Priority Data
May 27, 2013 (KR) 10-2013-0059708

(51) Int. Cl. A61K 31/785

(52) U.S. Cl. CPC A61K 31/785

(57) ABSTRACT

The present invention relates to a composition for dispersing or hydrating mucus, which comprises poly-gamma-glutamic acid, a biocompatible natural polymer, and more particularly, to a composition for treating mucus hypersecretory disease, which comprises poly-gamma-glutamic acid or poly-gamma-glutamic acid nanogels. The composition according to the present invention is effective in treating mucus-related diseases by effectively dispersing and hydrating mucus which is excessively secreted in vivo and highly sticky.

### TECHNICAL FIELD

The present invention relates to a composition for dispersing or hydrating mucus, which comprises poly-gamma-glutamic acid, a biocompatible natural polymer, and more particularly, to a composition for treating mucus hypersecretory disease, which comprises poly-gamma-glutamic acid or poly-gamma-glutamic acid nanogels.

### BACKGROUND ART

Mucin, which is the major component of mucus, collectively refers to viscous substances secreted from animal exocrine glands, and is a complex glycoprotein. It is known that mucin exhibits useful effects in vivo by promoting the digestion of cellular proteins, and has the effects of protecting the stomach wall and neutralizing poison.

Organs in which mucin is present include the oral cavity, the nasal cavity, the larynx, the gastrointestinal tracts, the eyes, the anus, and the vagina, and the thickness thereof ranges from several nanometers to 170 micrometers. The structure of the mucin network is maintained by various bonds, including ionic bonds, hydrogen bonds, disulfide bonds, van der Waals bonds, and entanglement between mucin molecules. Studies on mucus adhesive polymers which are connected with such bonds have been actively conducted, and polymers strongly interacting with bonds present in the mucin layer have been mainly studied. The mucus layer generally has a complex porous network structure. It is known that and bacteria having a size of several micrometers generally cannot pass through the mucus layer, but penetrates a portion of the mucus layer, which was destroyed or is thin in thickness. Although antibodies having a size of about 10 nm and plasmid DNAs larger than the antibodies are able to pass through the mucus layer, these are difficult to pass through the mucus layer due to the degradation by many enzymes present in the mucus layer. Viruses having a size of 200 nm or less weakly interact with mucin, and thus can quickly pass through the mucus layer (Yang, M. et al., Angewandte Chemie International Edition, 50(11):2597-2600, 2011).

Among mucus-related diseases, cystic fibrosis (CF) is a genetic disease that attacks various organs of the body, including the lungs, the liver, the pancreas, the urinary system, the reproductive system, and sweat glands. In this disease, thick sticky mucus is produced in cells that require the production of normal mucus, resulting in a state in which salt and water contents are not easily controlled. Because sticky mucus coming from the lungs inhibits the migration of pathogenic bacteria, bacterial infection frequently occurs. In addition, it affects mucous glands in bronchi, abnormally thick sticky mucus is produced. Due to the thick mucus, airway obstruction, promotion of bacterial growth, inflammation and infection occur.

In addition, thick sticky mucus may cause simple chronic bronchitis, chronic obstructive bronchitis, chronic asthmatic bronchitis, emphysema, chronic obstructive pulmonary disease, etc. In the pancreas, sticky mucus inhibits the movement of pancreatic juice, causing digestive disorders and malabsorption of fat and fat-soluble vitamins. For these reasons, dystrophy can appear, and it is frequent in babies. In addition, due to cystic fibrosis, sinusitis, nasal polyps, esophagitis, pancreatitis, liver cirrhosis, rectal prolapsed, diabetes, sterility (particularly male sterility), etc, may occur.

The above-described diseases are associated with the accumulation and poor dispersion of sticky mucus, the formation of abnormal mucin structures, and the impaired clearance of the mucous layer. In other words, these diseases are phenomena caused by the excessive secretion and viscosity of mucus. Thus, if the excessive secretion and viscosity of mucus can be reduced, the treatment of the above-described diseases can be promoted. Conventional therapeutic agents are mostly drugs that lyse mucin, but have been reported to have many side effects (Rubin, B. K. et al., Paediatric Respiratory Reviews, 7 Suppl 1, S215-219, 2006).

Among these drugs, representative drugs include deoxyribonuclease and N-acetylcystein. However, these drugs have the problem of causing serious solubilization of airway mucus. For this reason, the development of new therapeutic agents is required from the following two viewpoints. The first viewpoint is to disperse aggregated mucin that is already present, and the second viewpoint is to hydrate a mucin matrix, which is newly formed, without modification of an existing mucin network. Previous researchers reported that removing or chelating calcium ions (Ca²⁺) results in the swelling, hydration and dispersion of mucin networks. A study conducted by Dr. Chin indicated that calcium ion chelators such as EDTA assist in dispersion of mucin in cystic fibrosis, but when mucin was treated with carboxylated polystyrene, the dispersion of aggregated mucin increased (Chen, E. Y. et al., Scientific Reports, Vol. 2 Article number 211, 2012), and the effect of the carboxylated polystyrene is greater as the size thereof decreases. However, non-degradable synthetic polymers such as polystyrene have many problems when they are used as drugs in vivo.

Accordingly, the present inventors have made extensive efforts to develop biopolymers exhibiting the effects of dispersing and hydrating mucin, and as a result, have found that poly-gamma-glutamic acid has the effects of dispersing and hydrating mucin that is excessively secreted and aggregated in vivo, thereby completing the present invention.

### DISCLOSURE OF INVENTION

### Technical Problem

It is an object of the present invention to provide a composition for dispersing or hydrating mucus, which comprises a biopolymer.

Another object of the present invention is to provide a composition for treating mucus hypersecretory disease, which comprises a biopolymer.

Still another object of the present invention is to provide a method of dispersing or hydrating mucus using a biopolymer.

### Technical Solution

To achieve the above objects, the present invention provides a composition for dispersing or hydrating mucus, which comprises poly-gamma-glutamic acid or poly-gamma-glutamic acid nanogels.

The present invention also provides a composition for treating mucus hypersecretory disease, which comprises poly-gamma-glutamic acid or poly-gamma-glutamic acid nanogels.

The present invention also provides a method for treating mucus hypersecretory disease using poly-gamma-glutamic acid or poly-gamma-glutamic acid nanogels.

The present invention also provides a method for dispersing or hydrating mucus, which comprises treating aggregated mucus with poly-gamma-glutamic acid or poly-gamma-glutamic acid nanogels.

Other features and embodiments of the present invention will be more apparent from the following detailed descriptions and the appended claims.

### BRIEF DESCRIPTION OF THE DRAWINGS

FIG. 1 shows the hydrodynamic sizes of poly-gamma-glutamic acids having various molecular weights (50 kDa, 500 kDa and 500 kDa) thereof.

FIG. 2 shows the mucus dispersion and hydration properties of 50 kDa-poly-gamma-glutamic acid and nanogels.

FIG. 3 shows the mucus dispersion and hydration properties of 500 kDa-poly-gamma-glutamic acid and nanogels.

FIG. 4 shows the mucus dispersion and hydration properties of 5000 kDa-poly-gamma-glutamic acid and nanogels.

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods, which will be described below, are those well known and commonly employed in the art.

In one aspect, the present invention is directed to a composition for dispersing or hydrating mucus, which comprises poly-gamma-glutamic acid or poly-gamma-glutamic acid nanogels, and to a method of dispersing or hydrating mucus using poly-gamma-glutamic acid or poly-gamma-glutamic acid nanogels.

Poly-gamma-glutamic acid that is used in the present invention is a polypeptide having a carboxyl group, and is produced using a salt-tolerant Bacillus subtilis Chungkookjang strain that produces high-molecular-weight poly-gamma-glutamic acid (Korean Patent No. 500796). In addition, patent applications relating to anticancer compositions, immune adjuvants, immune enhancers, and virus infection inhibitors, which contain poly-gamma-glutamic acid, have been filed (Korean Patent Nos. 496606, 517114, 475406, and 873179). Further, as studies on the medicinal use of substances have been continuously conducted, the various effects of substances have been continuously found.

Poly-gamma-glutamic acid is rich in carboxyl groups (COO—). Thus, treatment of excessively sticky mucus with poly-gamma-glutamic acid exhibits the effects of swelling, hydrating and dispersing the mucus, and can cause electrostatic repulsion between calcium ion chelators and the anionic groups of mucin, such as sialic, sulfate and carboxyl functional groups.

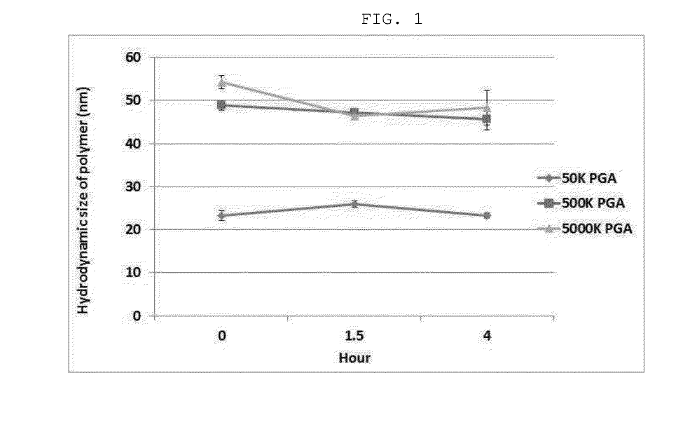

In the present invention, poly-gamma-glutamic acids having molecular weights of 50 kDa, 500 kDa and 5000 kDa were tested on their effects on the dispersion and hydration of mucus. As shown in FIGS. 1 to 4, when poly-gamma-glutamic acid was used at a concentration of 1 mg/ml, it could be seen that the size of mucin was greatly reduced. In addition, when mucus was treated with various concentrations (1 mg/mL, 500 μg/mL and 10 μg/mL) of poly-gamma-glutamic acid-cholesterol nanogels, it could be seen that, as the concentration of the poly-gamma-glutamic acid nanogels increased, the effect of the poly-gamma-glutamic acid nanogels on the dispersion of mucin increased.

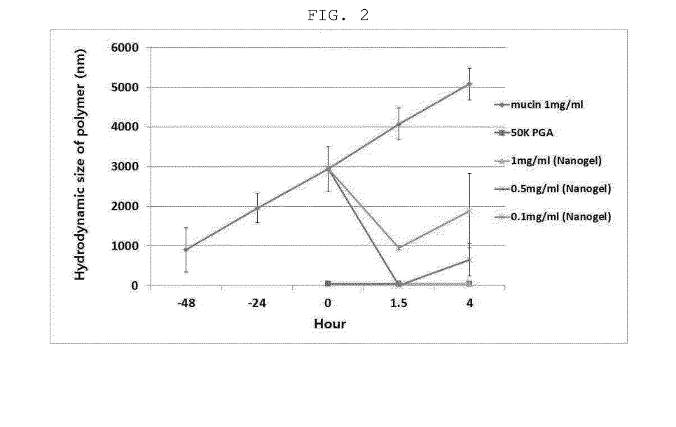

In an example of the present invention, poly-gamma-glutamic acid-cholesterol nanogels prepared by reacting poly-gamma-glutamic acid with cholesterol-amine were tested for their effects on the dispersion and hydration of mucus. As a result, as shown in FIGS. 2 to 4, it could be seen that mucin was dispersed and hydrated, and thus the size thereof was greatly reduced.

In another aspect, the present invention is directed to a composition for treating mucus hypersecretory disease, which comprises poly-gamma-glutamic acid or poly-gamma-glutamic nanogels.

The ability of poly-gamma-glutamic acid or poly-gamma-glutamic nanogels to disperse and hydrate mucus can be used to treat and alleviate diseases caused by excessive mucus secretion.

In still another aspect, the present invention is directed to a method of treating mucus hypersecretory disease using poly-gamma-glutamic acid or poly-gamma-glutamic nanogels.

As used herein, the term “mucus” refers to a mucus present in the mucus layer of oral cavity mucosa, nasal cavity mucosa, respiratory system mucosa, eye mucosa, reproductive system mucosa, skin ulcer sites or the like.

In cystic fibrosis (CF), thick sticky mucus is produced in cells of various organs of the body, including the lungs, the liver, the pancreas, the urinary system, the reproductive system, and sweat glands, resulting in a state in which salt and water contents are not easily controlled. Due to cystic fibrosis, sinusitis, nasal polyps, esophagitis, pancreatitis, liver cirrhosis, rectal prolapsed, diabetes, sterility (particularly male sterility), etc, may secondarily occur.

When poly-gamma-glutamic acid or poly-gamma-glutamic nanogels are administered to cystic fibrosis patients, excessively secreted mucus can be dispersed and hydrated, and thus the inconvenience of the patients can be reduced and the development of the secondary diseases can also be prevented.

In addition, excessive secretion of mucus can cause simple chronic bronchitis, chronic obstructive bronchitis, chronic asthmatic bronchitis, emphysema, and chronic obstructive pulmonary disease. In the pancreas, excessive mucus secretion inhibits the movement of pancreatic juice, causing digestive disorders and malabsorption of fat and fat-soluble vitamins.

In the present invention, examples of the mucus hypersecretory disease include cystic fibrosis (CF), chronic obstructive pulmonary disease (COPD), simple chronic bronchitis, chronic obstructive bronchitis, chronic asthmatic bronchitis, emphysema, chronic obstructive pulmonary disease, and digestive disease, but are not limited thereto.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1

### Synthesis of Poly-Gamma-Glutamic Acid Nanogels

To synthesize cholesterol-amine, ethylenediamine (250 mmol) was dissolved in 250 ml of toluene, and 2.25 g of cholesterol was dissolved in 50 ml of toluene and added dropwise to the ethylenediamine solution over 10 minutes. Immediately after addition, the mixed solution was allowed to react with stirring at room temperature for 16 hours. After completion of the reaction, the reaction solution was rinsed several times with deionized water, and the clear organic layer was dried with magnesium sulfate. The dried solution was rotary-evaporated to remove toluene, rinsed several times with a mixture of 20 ml of dichloromethane and 20 ml of methanol, and then filtered through a 1.0-μm PTFE filter, thereby preparing cholesterol-amine as a white solid.

1 g of poly-gamma-glutamic acid (Bioleaders, Korea) was dissolved in 10 ml of DMSO at 40° C. for about one day, and 1 g of the cholesterol-amine (obtained from Chungnam National University, Korea) prepared as described above was dissolved in 10 ml of tetrahydrofuran (THF, Sigma Aldrich, USA). The cholesterol-amine solution was slowly added dropwise to the poly-gamma-glutamic acid solution, and 1 g of carbodiimide (Sigma Aldrich, USA) was added to the mixture solution which was then allowed to react at 40° C. for about one day. The reaction solution was cooled to room temperature, and then rotary-evaporated to remove THF. The resulting solution was precipitated in acetone, and the solvent and the solute were sufficiently separated from each other by centrifugation to thereby remove acetone, and the remaining material was dried at 40° C. The dried sample was added to deionized water, and sodium hydrogen carbonate was added to the sample in the same molar amount as the poly-gamma-glutamic-acid used in the reaction to thereby neutralize the poly-gamma-glutamic-acid. The resulting solution was stirred with 5 g of amberlite (Sigma-Aldrich, USA) for about 2 hours to remove unreacted cholesterol and impurities. The stirred solution was filtered through a mesh to remove amberlite and was dialyzed for 2 days using a cellulose membrane tube (MWCO 12,000, Sigma-Aldrich, USA). The dialyzed solution was freeze-dried to obtain nanomicelles. The nanomicelles were analyzed by NMR to measure the amount of cholesterol introduced. The results of NMR analysis indicated that 1.7 mol % of cholesterol was bonded.

### Example 2

### Experiment on the Effects of Poly-Gamma-Glutamic Acid on Mucus Hydration and Dispersion

An experiment on the effects of poly-gamma-glutamic acid on mucus hydration and dispersion was performed in the following manner. First, porcine gastric mucin (Sigma-Aldrich, USA) was completely dissolved in a buffer (1.2 mM Ca²⁺, 20 mM Tris-HCl, 10 mM MES with HBSS) at a concentration of 1 mg/L. The solution was washed with 0.1N HCl and filtered through a 0.2 μm PES membrane filter. The filtrate was allowed to stand for 48 hours so that the mucin would aggregate, and then the size of the mucin was measured by DLS (dynamic light scattering, Otusk, Japan) with time.

After 48 hours, each of poly-gamma-glutamic acid (Bioleaders, Korea) and the poly-gamma-glutamic acid nanogels (prepared in Example 1), which have molecular weights of 50, 500 and 5000 kDa, was added to the mucin at concentrations of 1 mg/mL, 500 μg/mL and 100 μg/mL, and after 0 hr, 1.5 hrs and 4 hrs, the hydrodynamic sizes of the polymer poly-gamma-glutamic acid and the poly-gamma-glutamic acid nanogels were measured by DLS.

As a result, as shown in FIG. 1, it could be seen that the hydrodynamic sizes of the poly-gamma-glutamic acids having various molecular weights (50 kDa, 500 kDa and 5000 kDa) increased as the molecular weight increased.

Referring to FIG. 2, it can be seen that the hydrodynamic size of the mucus layer polymer (1 mg/ml) increased with the passage of time, suggesting that gradual aggregation of the mucus layer occurred. However, when 50 kDa-poly-gamma-glutamic acid was added at a concentration of 0.1 mg/ml, it could be seen that the hydrodynamic size thereof was small such that it could not be measured, suggesting that the aggregated mucus layer was hydrated or dispersed.

In addition, the poly-gamma-glutamic acid nanogels was added at a concentration of about 0.1-0.5 mg/ml, the size of the mucus layer was greatly reduced after about 1.5 hours, and the size increased again after 4 hours, suggesting that re-aggregation of the mucus layer occurred. However, at a concentration of about 1 mg/ml or higher, the mucus layer was maintained in a hydrated and dispersed state even with the passage of time, and thus the size thereof could not be measured.

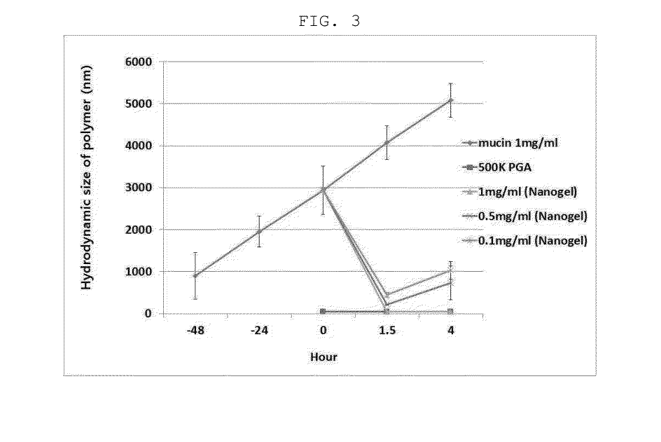

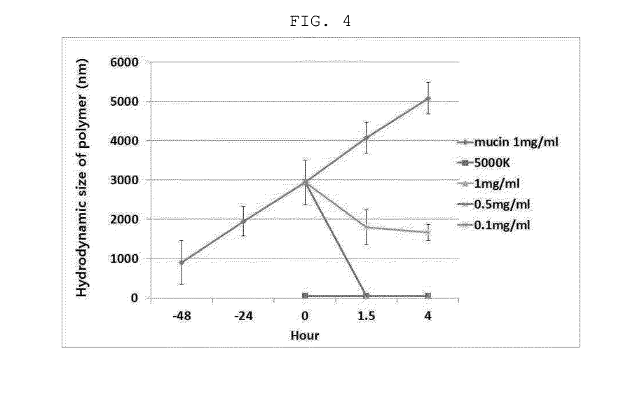

FIGS. 3 and 4 show the mucus dispersion and hydration properties of poly-gamma-glutamic acid and poly-gamma-glutamic acid nanogels, which have increased molecular weights of 500 kDa and 5000 kDa. As can be seen therein, the results similar to those as described above were shown. When the poly-gamma-glutamic acid was used at a concentration of 1 mg/ml, it could be seen that the size of mucin was greatly reduced. In addition, when the poly-gamma-glutamic acid-cholesterol nanogels were used at varying concentrations (1 mg/mL, 500 μg/mL, and 10 μg/mL), it could be seen that, as the concentration of the poly-gamma-glutamic acid nanogels increased, the effect of the poly-gamma-glutamic acid nanogels on the dispersion of mucin increased.

### INDUSTRIAL APPLICABILITY

As described above, the composition according to the present invention is effective in treating mucus-related diseases by effectively dispersing and hydrating mucus which is excessively secreted in vivo and highly sticky.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for dispersing or hydrating mucus, which comprises administering to a mammal in need thereof a composition comprising poly-gamma-glutamic acid or poly-gamma-glutamic acid nanogels.

2. The method of claim 1, wherein the poly-gamma-glutamic acid has a molecular weight of 1-15,000 kDa.

3. The method of claim 1, wherein the poly-gamma-glutamic acid nanogels are poly-glutamic acid-cholesterol polymer nanogels.

4. A method for treating mucus hypersecretory disease in a mammal, which comprises administering to the mammal a composition comprising poly-gamma-glutamic acid or poly-gamma-glutamic acid nanogels.

5. The method of claim 4, wherein the poly-gamma-glutamic acid has a molecular weight of 1-15,000 kDa.

6. The method of claim 4, wherein the poly-gamma-glutamic acid nanogels are poly-glutamic acid-cholesterol polymer nanogels.

7. The method of claim 4, wherein the mucus hypersecretory disease is selected from the group consisting of cystic fibrosis (CF), chronic obstructive pulmonary disease (COPD), simple chronic bronchitis, chronic obstructive bronchitis, chronic asthmatic bronchitis, emphysema, chronic obstructive pulmonary disease, and digestive disease.
